(19) United States
(12) Patent Application Publication
(10) Pub. No.: US 2021/0231659 A1
(43) Pub. Date: Jul. 29, 2021

(54) DETECTION AND ISOLATION OF MYELOID-DERIVED SUPPRESSOR CELL SUBPOPULATIONS

(71) Applicants: Scripps Health, San Diego, CA (US); The Scripps Research Institute, La Jolla, CA (US)

(72) Inventors: Darren SIGAL, San Diego, CA (US); Matthew MACAULEY, Edmonton (CA)

(21) Appl. No.: 17/165,718

(22) Filed: Feb. 2, 2021

Related U.S. Application Data
(63) Continuation of application No. PCT/US2019/044676, filed on Aug. 1, 2019.
(60) Provisional application No. 62/714,512, filed on Aug. 3, 2018.

(51) Int. Cl. G01N 33/569; G01N 33/574

(52) U.S. Cl. CPC G01N 33/56966; G01N 2800/52; G01N 33/57492

(57) ABSTRACT

Myeloid derived suppressor cells (MDSCs) are a heterogeneous group of immature myeloid cells with the ability to mediate immunosuppression in cancer. Disclosed herein are methods of identifying MDSCs, methods of isolating MDSCs, and methods of treating patients.

### CROSS-REFERENCE

This application is a continuation of International Application No. PCT/US2019/044676, filed on Aug. 1, 2019, which claims the benefit of U.S. Provisional Patent Application No. 62/714,512 filed on Aug. 3, 2018, Entitled: “Detection And Isolation Of Myeloid-Derived Suppressor Cell Subpopulations,” which are incorporated herein by reference in their entirety.

### BACKGROUND OF THE DISCLOSURE

Myeloid-derived suppressor cells (MDSCs) are a heterogeneous population of cells recruited to the tumor microenvironment with the ability to suppress T-cell responses. MDSCs therefore serve as an attractive target for the detection and monitoring of cancer. However, before MDSCs are used to monitor or diagnose cancer, methods are needed to distinguish MDSCs from other immune cells, such as neutrophils and monocytes, and to distinguish specific subpopulations of MDSCs most relevant in cancer.

### SUMMARY OF THE DISCLOSURE

Disclosed herein, in some embodiments, is a method of identifying a population of myeloid-derived suppressor cells (MDSCs) in a biological sample, comprising: detecting cells from a biological sample comprising (i) high levels of a neutrophil biomarker; (ii) low levels of a monocyte biomarker; (iii) low levels of CD16; and (iv) low levels of Siglec-9. In some embodiments, the method further comprises detecting cells comprising low levels of Siglec-5. In some embodiments, the method further comprises detecting cells comprising high levels of CD33 (Siglec-3). In some embodiments, the method further comprises detecting cells comprising low levels of Siglec-5 and high levels of CD33 (Siglec-3). In some embodiments, the neutrophil biomarker comprises CD15. In some embodiments, the monocyte biomarker comprises CD14. In some embodiments, the method further comprises detecting cells comprising low levels of an eosinophil biomarker, wherein the eosinophil biomarker is Siglec-8. In some embodiments, the method further comprises detecting cells comprising low levels of a basophil biomarker, wherein the basophil biomarker is CD123. In some embodiments, the method further comprises detecting cells comprising low levels of lymphocyte biomarkers. In some embodiments, the lymphocyte biomarkers comprise CD3, CD19, CD56, or a combination thereof. In some embodiments, the high levels are a level of expression above a threshold level of expression and the low levels are a level of expression below a threshold level of expression. In some embodiments, the biological sample is a blood sample. In some embodiments, the blood sample is whole blood or a buffy coat. In some embodiments, the biological sample is a tissue sample. In some embodiments, the population of MDSCs is detected using an antibody or antigen-binding fragment thereof. In some embodiments, the population of MDSCs is detected using flow cytometry. In some embodiments, the population of MDSCs is detected using an enzyme-linked immunosorbent assay (ELISA). In some embodiments, the population of MDSCs is detected using single cell analysis of cell surface biomarkers. In some embodiments, the population of MDSCs is detected using single cell RNA sequencing. In some embodiments, positive identification of the population of MDSCs is indicative of the presence of a cancer. In some embodiments, the cancer is a solid tumor. In some embodiments, the cancer is a cancer of the adrenal gland, bile duct (e.g., cholangiocarcinoma), bladder, blood (e.g., a leukemia, a lymphoma, multiple myeloma, acute myeloid leukemia, acute lymphoid leukemia, chronic myeloid leukemia, or chronic lymphoid leukemia), bone, brain, breast, cervix, colorectal system (e.g., colorectal cancer or colon cancer), esophagus, gallbladder, gastric system, head and neck, kidney, liver, lung, ovary, pancreas, prostate, reticuloendothelial system, salivary gland, skin (e.g., melanoma), small intestine, soft tissue, thymus, or uterus. In some embodiments, the cancer is a pancreatic cancer. In some embodiments, the cancer is a lung cancer. In some embodiments, the cancer is a colon cancer. In some embodiments, the cancer is a breast cancer. In some embodiments, the cancer is a gastric cancer. In some embodiments, the cancer is an esophageal cancer. In some embodiments, the cancer is an ovarian cancer. In some embodiments, the cancer is a uterine cancer. In some embodiments, the cancer is a prostate cancer. In some embodiments, the cancer is a bladder cancer. In some embodiments, the cancer is a liver cancer. In some embodiments, the cancer is a cholangiocarcinoma. In some embodiments, the cancer is a neuroendocrine tumor. In some embodiments, the cancer is a gastrointestinal stromal tumor. In some embodiments, the cancer is a sarcoma. In some embodiments, the cancer is a brain cancer. In some embodiments, the cancer is a skin cancer. In some embodiments, the cancer is a melanoma. In some embodiments, the cancer is a liquid tumor. In some embodiments, the cancer is a multiple myeloma. In some embodiments, the cancer is an acute myeloid leukemia. In some embodiments, the cancer is an acute lymphoid leukemia. In some embodiments, the cancer is a chronic myeloid leukemia. In some embodiments, the cancer is a chronic lymphoid leukemia. In some embodiments, the biological sample is from an individual at high risk of developing a cancer. In some embodiments, the biological sample is from an individual who has previously had a cancer and wherein positive identification of the myeloid-derived suppressor cell is indicative of recurrence of the cancer. In some embodiments, the biological sample is from an individual diagnosed with a cancer. In some embodiments, the individual is undergoing active surveillance or active therapy.

Also disclosed herein, in some embodiments, is a method of preparing a purified population of myeloid-derived suppressor cells (MDSCs) from a biological sample, the method comprising isolating a population of MDSCs comprising: (i) high levels of a neutrophil biomarker; (ii) low levels of monocyte biomarker; (iii) low levels of CD16; and (iv) low levels of Siglec-9. In some embodiments, the population of MDSCs further comprise low levels of Siglec-5. In some embodiments, the population of MDSCs further comprise high levels of CD33 (Siglec-3). In some embodiments, the population of MDSCs further comprise low levels of Siglec-5 and high levels of CD33 (Siglec-3). In some embodiments, the neutrophil biomarker comprises CD15. In some embodiments, the monocyte biomarker comprises CD14. In some embodiments, the population of MDSCs further comprise low levels of an eosinophil biomarker, wherein the eosinophil biomarker is Siglec-8. In some embodiments, the population of MDSCs further comprise low levels of a basophil biomarker, wherein the basophil biomarker is CD123. In some embodiments, the population of MDSCs further comprise low levels of lymphocyte biomarkers. In some embodiments, the lymphocyte biomarkers comprise CD3, CD19, CD56, or a combination thereof. In some embodiments, the high levels are a level of expression above a threshold level of expression and the low levels are a level of expression below a threshold level of expression. In some embodiments, the biological sample is a blood sample. In some embodiments, the blood sample is whole blood or a buffy coat. In some embodiments, the biological sample is a tissue sample. In some embodiments, the population of MDSCs is isolated using fluorescent activated cell sorting (FACS).

Also disclosed herein, in some embodiments, is a kit comprising an agent capable of detecting a neutrophil biomarker, an agent capable of detecting a monocyte biomarker, an agent capable of detecting CD16, and an agent capable of detecting Siglec-9. In some embodiments, the kit further comprises an agent capable of detecting Siglec-5. In some embodiments, the kit further comprises an agent capable of detecting CD33 (Siglec-3). In some embodiments, the kit comprises an agent capable of detecting Siglec-5 and CD33 (Siglec-3). In some embodiments, the agent capable of detecting the neutrophil biomarker comprises an antibody or antigen binding fragment thereof that binds to CD15. In some embodiments, the agent capable of detecting the monocyte biomarker comprises an antibody or antigen-binding fragment thereof that binds to CD14. In some embodiments, the agent capable of detecting CD16 comprises an antibody or antigen-binding fragment thereof that binds to CD16. In some embodiments, the agent capable of detecting Siglec-9 comprises an antibody or antigen-binding fragment thereof that binds to Siglec-9. In some embodiments, the agent capable of detecting Siglec-5 comprises an antibody or antigen-binding fragment thereof that binds to Siglec-5. In some embodiments, the agent capable of detecting CD33 (Siglec-3) comprises an antibody or antigen-binding fragment thereof that binds to CD33 (Siglec-3). In some embodiments, the agent capable of detecting Siglec-5 comprises an antibody or antigen-binding fragment thereof that binds to Siglec-5 and the agent capable of detecting CD33 (Siglec-3) comprises an antibody or antigen-binding fragment thereof that binds to CD33 (Siglec-3). In some embodiments, the kit further comprises an agent capable of detecting an eosinophil biomarker, wherein the eosinophil biomarker is Siglec-8. In some embodiments, the agent capable of detecting the eosinophil biomarker comprises an antibody or antigen-binding fragment thereof that binds to Siglec-8. In some embodiments, the kit further comprises an agent capable of detecting a basophil biomarker, wherein the basophil biomarker is CD123. In some embodiments, the agent capable of detecting the basophil biomarker comprises an antibody or antigen-binding fragment thereof that binds to CD123. In some embodiments, the kit further comprises an agent capable of detecting a lymphocyte biomarker. In some embodiments, the agent capable of detecting a lymphocyte biomarker comprises one or more antibodies or antigen-binding fragments thereof that bind to CD3, CD19, CD56, or a combination thereof.

Also disclosed herein, in some embodiments, is a method of treating a cancer in a patient in need thereof, comprising administering an anti-cancer therapy to the patient, wherein a biological sample from the patient has been identified as comprising a population of myeloid-derived suppressor cells (MDSCs) comprising: (i) high levels of a neutrophil biomarker; (ii) low levels of a monocyte biomarker; (iii) low levels of CD16; and (iv) low levels of Siglec-9. In some embodiments, the population of MDSCs further comprise low levels of Siglec-5. In some embodiments, the population of MDSCs further comprise high levels of CD33 (Siglec-3). In some embodiments, the neutrophil biomarker comprises CD15. In some embodiments, the monocyte biomarker comprises CD14. In some embodiments, the population of MDSCs further comprise low levels of an eosinophil biomarker, wherein the eosinophil biomarker is Siglec-8. In some embodiments, the population of MDSCs further comprise low levels of a basophil biomarker, wherein the basophil biomarker is CD123. In some embodiments, the population of MDSCs further comprise low levels of lymphocyte biomarkers. In some embodiments, the lymphocyte biomarkers comprise CD3, CD19, CD56, or a combination thereof. In some embodiments, the high levels are a level of expression above a threshold level of expression and the low levels are a level of expression below a threshold level of expression. In some embodiments, the biological sample is a blood sample. In some embodiments, the blood sample is whole blood or a buffy coat. In some embodiments, the biological sample is a tissue sample. In some embodiments, the method further comprises identifying the population of MDSCs from the biological sample of the patient. In some embodiments, the identifying the population of MDSCs comprises detecting using an antibody or antigen-binding fragment thereof. In some embodiments, the identifying the population of MDSCs comprises detecting using flow cytometry. In some embodiments, the identifying the population of MDSCs comprises detecting using an enzyme-linked immunosorbent assay (ELISA). In some embodiments, the identifying the population of MDSCs comprises detecting using single cell analysis of cell surface biomarkers. In some embodiments, the identifying the population of MDSCs comprises detecting using single cell RNA sequencing. In some embodiments, positive identification of the population of MDSCs is indicative of the presence of the cancer. In some embodiments, the patient is at high risk of developing the cancer. In some embodiments, the patient has previously had the cancer and wherein positive identification of the myeloid-derived suppressor cell is indicative of recurrence of the cancer. In some embodiments, the patient has been diagnosed with the cancer. In some embodiments, the patient is undergoing active surveillance or active therapy. In some embodiments, the cancer is a solid tumor. In some embodiments, the cancer is a cancer of the adrenal gland, bile duct (e.g., cholangiocarcinoma), bladder, blood (e.g., a leukemia, a lymphoma, multiple myeloma, acute myeloid leukemia, acute lymphoid leukemia, chronic myeloid leukemia, or chronic lymphoid leukemia), bone, brain, breast, cervix, colorectal system (e.g., colorectal cancer or colon cancer), esophagus, gallbladder, gastric system, head and neck, kidney, liver, lung, ovary, pancreas, prostate, reticuloendothelial system, salivary gland, skin (e.g., melanoma), small intestine, soft tissue, thymus, or uterus. In some embodiments, the cancer is a pancreatic cancer. In some embodiments, the cancer is a lung cancer. In some embodiments, the cancer is a colon cancer. In some embodiments, the cancer is a breast cancer. In some embodiments, the cancer is a gastric cancer. In some embodiments, the cancer is an esophageal cancer. In some embodiments, the cancer is an ovarian cancer. In some embodiments, the cancer is a uterine cancer. In some embodiments, the cancer is a prostate cancer. In some embodiments, the cancer is a bladder cancer. In some embodiments, the cancer is a liver cancer. In some embodiments, the cancer is a cholangiocarcinoma. In some embodiments, the cancer is a neuroendocrine tumor. In some embodiments, the cancer is a gastrointestinal stromal tumor. In some embodiments, the cancer is a sarcoma. In some embodiments, the cancer is a brain cancer. In some embodiments, the cancer is a skin cancer. In some embodiments, the cancer is a melanoma. In some embodiments, the cancer is a liquid tumor. In some embodiments, the cancer is a multiple myeloma. In some embodiments, the cancer is an acute myeloid leukemia. In some embodiments, the cancer is an acute lymphoid leukemia. In some embodiments, the cancer is a chronic myeloid leukemia. In some embodiments, the cancer is a chronic lymphoid leukemia. In some embodiments, the cancer is a pancreatic cancer. In some embodiments, the anti-cancer therapy is administered instead of a second anti-cancer therapy. In some embodiments, the anti-cancer therapy is administered in addition to a second anti-cancer therapy. In some embodiments, the second anti-cancer therapy has previously been administered to the patient. In some embodiments, the anti-cancer therapy has previously been administered to the patient. In some embodiments, a second biological sample from the patient has been identified as comprising the population of MDSCs. In some embodiments, the method further comprises modifying an amount of the anti-cancer therapy administered to the patient based on comparing a size of the population of MDSCs between the biological sample and the second biological sample. In some embodiments, the method further comprises changing the anti-cancer therapy administered to the patient based on comparing a size of the population of MDSCs between the biological sample and the second biological sample. In some embodiments, the anti-cancer therapy is a chemotherapy. In some embodiments, the anti-cancer therapy is an immunotherapy. In some embodiments, the anti-cancer therapy is a hormone therapy. In some embodiments, the anti-cancer therapy is a stem cell transplant. In some embodiments, the anti-cancer therapy is a radiation therapy. In some embodiments, the anti-cancer therapy is a surgery. In some embodiments, the anti-cancer therapy is a small molecule drug. In some embodiments, the anti-cancer therapy is an antibody or antigen-binding fragment thereof. In some embodiments, the anti-cancer therapy is a checkpoint inhibitor. In some embodiments, the anti-cancer therapy is a kinase inhibitor. In some embodiments, the anti-cancer therapy is a gene-editing therapy. In some embodiments, the anti-cancer therapy is a cellular therapy. In some embodiments, the cellular therapy is a chimeric antigen receptor (CAR)-T cell therapy or a transgenic T cell receptor (tg-TCR) T cell therapy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a scheme for cell purification from whole blood by density gradient centrifugation.

FIGS. 2A-2B depict a flow cytometry experiment demonstrating one method for identifying MDSCs. FIG. 2A demonstrates MDSCs detected in whole blood, granulocyte, and buffy coat samples from a healthy patient while FIG. 2B demonstrates MDSCs detected in a buffy coat sample from a pancreatic cancer patient.

FIG. 3 depicts a comparison between MDSCs detected in buffy coat samples from healthy patients versus pancreatic cancer patients.

FIGS. 4A-4D depict a flow cytometry experiment characterizing MDSC subpopulations in pancreatic cancer patients and healthy individuals. FIG. 4A demonstrates CD16^{low} and CD16^{high} MDSC subpopulations detected in whole blood, granulocyte, and buffy coat samples from a healthy patient. FIG. 4B demonstrates CD16^{low} and CD16^{high} MDSC subpopulations detected in whole blood, granulocyte, and buffy coat samples from a pancreatic cancer patient. FIG. 4C demonstrates LOX-1 levels observed in CD16^{low} and CD16^{high} MDSC subpopulations detected in whole blood, granulocyte, and buffy coat samples from a healthy patient. FIG. 4D demonstrates LOX-1 levels observed in CD16^{low} and CD16^{high} MDSC subpopulations detected in whole blood, granulocyte, and buffy coat samples from a pancreatic cancer patient.

FIGS. 5A-5B depict a comparison of MDSCs in healthy patients compared to pancreatic cancer patients. FIG. 5A depicts the percentage of CD16^{low}/Siglec9^{low} MDSCs observed in an MDSC population while FIG. 5B depicts the number of CD16^{low}/Siglec9^{low} MDSCs observed per mL of whole blood.

FIG. 6 depicts a comparison of Siglec-3, Siglec-5, and Siglec-9 expression levels in CD16^{high} versus CD16^{low} MDSCs.

FIG. 7 illustrates a workflow for the sorting and functional analysis of MDSC subpopulations.

FIGS. 8A-8C depict T-cell proliferation experiments using CD16^{high} and CD16^{low} MDSCs derived from pancreatic cancer patients and healthy individuals. FIG. 8A illustrates CD8⁺ T-cell proliferation when incubated in the presence of CD16^{high} MDSCs from a healthy patient and CD16^{low} MDSCs from a pancreatic cancer patient. FIG. 8B illustrates CD4⁺ T-cell proliferation when incubated in the presence of CD16^{high} MDSCs from a healthy patient and CD16^{low} MDSCs from a pancreatic cancer patient at a 1:1 ratio. FIG. 8C illustrates CD4⁺ T-cell proliferation when incubated in the presence of CD16^{high} and CD16^{low} MDSCs from a healthy patient or pancreatic cancer patient in a 1:3 ratio.

### DETAILED DESCRIPTION OF THE DISCLOSURE

Myeloid derived suppressor cells (MDSCs) are a heterogeneous group of cells that expand during cancer, inflammation, and infection. In some embodiments, MDSCs comprise precursors for granulocytes, precursors for macrophages, precursors for dendritic cells (DCs), or a combination thereof. In some embodiments, the MDSC is a polymorphonuclear (PMN) MDSC or a monocytic MDSC. In some embodiments, MDSCs mediate immunosuppression in cancer, wherein anti-tumor immune responses are inhibited. In some embodiments, MDSCs stimulate tumor growth. In some embodiments, MDSCs suppress T cell responses. In some embodiments, the T-cells are CD8⁺ T-cells. In other embodiments, the T-cells are CD4⁺ T-cells. In some embodiments, the T cells are introduced as part of a therapy, e.g., T cells with chimeric antigen receptors (CAR-T cells) or transgenic T cell receptors.

In some embodiments, an MDSC is identified by the presence or expression level of a biomarker. In some embodiments, the biomarker is expressed on the surface of the MDSC. In some embodiments, the biomarker of the MDSC is expressed intracellularly. In some embodiments, the biomarker is a protein, a DNA encoding the protein, or an RNA encoding the protein. In some embodiments, the RNA is messenger RNA (mRNA). In some embodiments, the protein is a protein in the Sialic acid-binding Ig-like lectin (Siglec) family. In some embodiments, a subpopulation of MDSCs is identified by detection of at least one biomarker characterizing the subpopulation of MDSCs. In some embodiments, a subpopulation of MDSCs is identified by relatively higher detection of at least one biomarker. In some embodiments, a subpopulation of MDSCs is identified by relatively higher detection of at least one biomarker and relatively lower detection of at least one biomarker. In some embodiments, a subpopulation of MDSCs is identified by relatively lower detection of at least one biomarker. In some embodiments, the subpopulation of MDSCs is a subpopulation of MDSCs associated with a cancer. In some embodiments, an MDSC subpopulation is identified by relatively higher expression of one, two, three, four, five, six, seven, eight, nine or ten biomarkers. In some embodiments, an MDSC subpopulation is identified by relatively lower expression of one, two three, four, five, six, seven, eight, nine, or ten biomarkers. In some embodiments, an MDSC subpopulation is identified by relatively higher expression of one, two, three, four, five, six, seven, eight, nine or ten biomarkers, and relatively lower expression of one, two three, four, five, six, seven, eight, nine, or ten biomarkers. Methods for identification and/or isolation of myeloid-derived suppressor cells (MDSCs)

Disclosed herein are methods of identifying a myeloid-derived suppressor cell (MDSC) (or population of MDSCs) in a biological sample, as well as methods of preparing a purified population of myeloid-derived suppressor cells (MDSCs) from a biological sample. Also disclosed herein, in certain embodiments, are methods of identifying an MDSC (or population of MDSCs) in a biological sample, comprising: detecting cells from a biological sample comprising (i) high levels of a neutrophil biomarker; (ii) low levels of monocyte biomarker; (iii) low levels of CD16; and (iv) low levels of Siglec-9. In some embodiments, the neutrophil biomarker is a high level of CD15. In some embodiments, the monocyte biomarker is a low level of CD14.

In some embodiments, the biological sample is a blood sample. In some embodiments, the blood sample is a peripheral blood sample. In some embodiments, the peripheral blood sample is a whole blood sample. In some embodiments, the biological sample is a tissue sample. In some embodiments, the biological sample is a cancer tissue sample (e.g., a biopsy). In some embodiments, the biological sample is a non-cancer tissue sample. In some embodiments, the peripheral blood sample is a buffy coat sample. In some embodiments, the biological sample is taken from an individual. In some embodiments, the individual is a human. In some embodiments, the individual is a mammal. In some embodiments, the mammal is a human, non-human primate, dog, cat, rabbit, mouse, or rat. In some embodiments, the mammal is a human. In some embodiments, the individual is diagnosed with cancer. In some embodiments, the individual is at risk of developing cancer. In some embodiments, the individual is in remission from cancer. In some embodiments, the individual is undergoing therapy or surveillance for cancer. In some embodiments, the cancer is a cancer of the adrenal gland, bile duct (e.g., cholangiocarcinoma), bladder, blood (e.g., a leukemia, a lymphoma, multiple myeloma, acute myeloid leukemia, acute lymphoid leukemia, chronic myeloid leukemia, or chronic lymphoid leukemia), bone, brain, breast, cervix, colorectal system (e.g., colorectal cancer or colon cancer), esophagus, gallbladder, gastric system, head and neck, kidney, liver, lung, ovary, pancreas, prostate, reticuloendothelial system, salivary gland, skin (e.g., melanoma), small intestine, soft tissue, thymus, or uterus. In some embodiments, the cancer is a neuroendocrine tumor. In some embodiments, the cancer is a gastrointestinal stromal tumor. In some embodiments, the cancer is a sarcoma. In some embodiments, the cancer is pancreatic cancer. In some embodiments, the pancreatic cancer is a pancreatic adenocarcinoma. In some embodiments, the pancreatic cancer is a pancreatic endocrine tumor (PET).

In some embodiments, the biological sample comprises MDSCs, neutrophils, monocytes, eosinophils, basophils, red blood cells, lymphocytes, or a combination thereof. In some embodiments, the MDSC is a polymorphonuclear (PMN) MDSC or a monocytic MDSC. In some embodiments, the method comprises centrifugation of the biological sample. In some embodiments, Ficoll® is added to the biological sample prior to centrifugation. In some embodiments, the Ficoll® is Ficoll®-Paque. In some embodiments, centrifugation of a biological sample comprising a blood sample produces a first layer, a buffy coat, and a second layer. In some embodiments, the first layer comprises plasma. In some embodiments, the second layer comprises granulocytes. In some embodiments, the second layer comprises red blood cells, neutrophils, eosinophils, or a combination thereof. In some embodiments, the buffy coat comprises the mononuclear layer, including: lymphocytes, monocytes, basophils, MDSCs, or a combination thereof.

In some embodiments, isolating an MDSC comprises identifying MDSCs, MDSC subpopulations, non-MDSCs, or a combination thereof. In some embodiments, isolating an MDSC comprises isolating MDSCs, MDSC subpopulations, non-MDSCs, or a combination thereof. In some embodiments, non-MDSCs are selected from the group consisting of: lymphocytes, basophils, eosinophils, and any combination thereof. In some embodiments, non-MDSCs are selected from the group consisting of: lymphocytes, monocytes, basophils, red blood cells, neutrophils, eosinophils, and any combination thereof. In some embodiments, non-MDSCs are any cells that are not MDSCs.

In some embodiments, a biomarker is used to identify an MDSC or MDSC subpopulation. In some embodiments, a biomarker is used to separate or isolate an MDSC or MDSC subpopulation from non-MDSCs in a biological sample. In some embodiments, a biomarker is used to separate or isolate an MDSC subpopulation from a second MDSC subpopulation in a biological sample.

In some embodiments, a biomarker is used to identify a non-MDSC. In some embodiments, a biomarker is used to separate or remove non-MDSCs from MDSC subpopulations in a biological sample. In some embodiments, the biomarker used to identify, separate, and/or remove a non-MDSC is a biomarker identifying a lymphocyte, basophil, eosinophil, or a combination thereof. In some embodiments, the biomarker identifying a lymphocyte is a high level of CD3 (T-cells), a high level of CD19 (B-cells), a high level of CD56 (NK cells), or a combination thereof. In some embodiments, the biomarker identifying a basophil is a high level of CD123. In some embodiments, the biomarker identifying an eosinophil is a high level of Siglec-8.

In some embodiments, the MDSC, MDSC subpopulation, or non-MDSC is identified using flow cytometry, mass cytometry, immunomagnetic sorting, ELISA, multiplex immunoassay, western blot, protein microarray, mass spectrometry, sequencing, or a combination thereof. In some embodiments, the MDSC, MDSC subpopulation, or non-MDSC is identified with a detectable probe. In some embodiments, the detectable probe is an antibody or antigen-binding fragment thereof, an aptamer, a magnetic bead, a fluorophore, a fluorescent protein, or a combination thereof. In some embodiments, the detectable probe binds to the biomarker used to identify the MDSC, MDSC subpopulation, or non-MDSC.

In some embodiments, the MDSC, MDSC subpopulation, or non-MDSC is identified using flow cytometry. In some embodiments, the method comprises subjecting a sample to flow cytometry to identify non-MDSCs. In some embodiments, the method comprises subjecting a sample to flow cytometry to identify MDSCs. In some embodiments, the method comprises subjecting a sample to flow cytometry to identify MDSC subpopulations.

In some embodiments, MDSCs and/or non-MDSCs are isolated using cell sorting. In some embodiments, the cell sorting is fluorescent activated cell sorting (FACS). In some embodiments, the cell sorting is magnetic activated cell sorting (MACS). In some embodiments, cell sorting isolates a cell based on the presence or absence of a detectable probe. In some embodiments, the detectable probe is a fluorescent marker. In some embodiments, the detectable probe is a magnetic probe. In some embodiments, the detectable probe is an isotopic probe. In some embodiments, the method comprises subjecting a sample to cell sorting to remove MDSCs, MDSC subpopulations, non-MDSCs, or a combination thereof. In some embodiments, the method comprises subjecting a sample to cell sorting to isolate MDSCs, MDSC subpopulations, non-MDSCs, or a combination thereof. In some embodiments, the method comprises subjecting a sample to cell sorting to remove MDSCs, MDSC subpopulations, non-MDSCs, or a combination thereof, and to isolate MDSCs, MDSC subpopulations, non-MDSCs, or a combination thereof, In some embodiments, the method comprises subjecting a sample where non-MDSCs have been removed to flow cytometry to select for MDSCs or MDSC subpopulations.

In some embodiments, the detectable probe binds to a biomarker identifying a non-MDSC (e.g., based on high or low expression of the biomarker). In some embodiments, the biomarker identifying the non-MDSC is selected from at least one of the following: Siglec-8, CD123, CD3, CD19, CD56, and a combination thereof. In some embodiments, the detectable probe binds to a biomarker identifying an MDSC or MDSC subpopulation (e.g., based on high or low expression of the biomarker). In some embodiments, the biomarker utilized to identify the MDSC or MDSC subpopulation is selected from at least one of the following: CD14, CD15, CD16, Siglec-3 (CD33), Siglec-5, Siglec-9, and a combination thereof. In some embodiments, the biomarkers used to identify the MDSC or MDSC subpopulation is selected from at least one of the following: a low level of CD14, a high level of CD15, a low level of CD16, a low level of Siglec-9, a high level of Siglec-3 (CD33), a low level of Siglec-5, and a combination thereof

In some embodiments, the detectable probe comprises an antibody or antigen-binding fragment thereof conjugated to a fluorophore or fluorescent protein. In some embodiments, the detectable probe is an aptamer conjugated to a fluorophore. In some embodiments, the antibody, antigen-binding fragment thereof, or aptamer is an antibody, antigen-binding fragment thereof, or aptamer specific to the biomarker used to identify the MDSC, MDSC subpopulation, or non-MDSC (e.g., based on high or low expression of the biomarker by the MDSC, MDSC subpopulation, or non-MDSC). In some embodiments, the fluorophore is a xanthene, cyanine, squaraine, naphthalene, coumarin, oxadiazole, anthracene, pyrene, oxazine, acridine, arylmethine, tetrapyrrole, or a derivative thereof. In some embodiments, the xanthene derivative is a fluorescein, rhodamine, Oregon green, eosin, or Texas red. In some embodiments, the cyanine derivative is indocarbocyanine, oxacarbocyanine, thiacarbocyanine, or merocyanine. In some embodiemnts, the squaraine is Seta, SeTau, or Square dyes. In some embodiments, the oxadiazole derivative is pyridyloxazole, nitrobenzoxadiazole, or benzoxadiazole. In some embodiments, the anthracene derivative is an anthraquinone. In some embodiments, the pyrene derivative is cascade blue. In some embodiments, the oxazine derivative is nile red, nile blue, cresyl violet, or oxazine 170. In some embodiments, the acridine derivative is proflavin, acridine orange, or acridine yellow. In some embodiments, the arylmethine derivative is auramine, crystal violet, or malachite green. In some embodiments, the tetrapyrrole derivative is porphin, phthalocyanine, or bilirubin. In some embodiments, fluorophore is a commercially available fluorophore. In some embodiments, the commercially available fluorophore is a fluorophore in a family selected from Alexa Fluor®, DyLight®, HiLyte™, BODIPY®, FluoProbes®, Abberior®, Brilliant Violet™ families.

In some embodiments, the detectable probe comprises a fluorescent protein (FP). In some embodiments, the fluorescent protein is a monomer, a dimer, or a tetramer. In some embodiments, the fluorescent protein is a photoactivatable fluorescent protein. In some embodiments, any suitable fluorescent protein is used. Examples of fluorescent proteins include, but are not limited to, a green fluorescent protein (GFP), a cyan fluorescent protein (CFP), a yellow fluorescent protein (YFP), a red fluorescent protein (RFP), a Verde fluorescent protein (VFP), a kindling fluorescent protein (KFP), or mCHERRY.

In some embodiments, the MDSC, MDSC subpopulation, or non-MDSC is identified or isolated using magnetic activated cell sorting (MACS). In some embodiments, MACS detects or isolates a cell based on the presence of a detectable probe (e.g., via positive or negative selection). In some embodiments, the detectable probe comprises a magnetic bead. In some embodiments, the detectable probe comprises an antibody or antigen-binding fragment thereof conjugated to a magnetic particle. In some embodiments, the detectable probe comprises an aptamer conjugated to a magnetic particle. In some embodiments, the antibody, antigen-binding fragment thereof, or aptamer is an antibody, antigen-binding fragment thereof, or aptamer specific to the biomarker used to identify the MDSC or non-MDSC.

In some embodiments, fluorescent assisted cell sorting (FACS) is used to remove at least 0.05%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, or 99% of non-MDSCs, MDSCs, or a subpopulation of MDSCs from the biological sample. In some embodiments, flow cytometry is used to isolate at least 0.05%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, or 99% of the non-MDSCs, MDSCs, or a subpopulation of MDSCs from the biological sample. In some embodiments, flow cytometry is used to remove at least 5% of the non-MDSCs from the biological sample. In some embodiments, flow cytometry is used to remove at least 15% of the non-MDSCs from the biological sample. In some embodiments, flow cytometry is used to remove at least 40% of the non-MDSCs from the biological sample. In some embodiments, flow cytometry is used to remove at least 80% of the non-MDSCs from the biological sample. In some embodiments, flow cytometry is used to isolate at least 15% of the cells from the biological sample. In some embodiments, flow cytometry is used to isolate at least 40% of the cells from the biological sample. In some embodiments, flow cytometry is used to isolate at least 80% of the cells from the biological sample. In some embodiments, flow cytometry is used to isolate at least 98% of the cells from the biological sample. In some embodiments, the detectable probe is removed from the MDSC, MDSC subpopulation, or non-MDSC after the identifying. In some embodiments, the detectable probe is removed from the MDSC, MDSC subpopulation, or non-MDSC after the isolating.

In some embodiments, the MDSC, MDSC subpopulation, or non-MDSC is identified using immunomagnetic sorting (e.g., immunomagnetic sorting using positive and/or negative selection). In some embodiments, the MDSC, MDSC subpopulation, or non-MDSC is isolated using immunomagnetic sorting. In some embodiments, the immunomagnetic sorting is MACS. In some embodiments, the immunomagnetic sorting comprises: (a) binding a magnetic probe to a biomarker expressed on an MDSC, MDSC subpopulation, or non-MDSC in a sample; (b) applying a magnetic field to the sample to separate an MDSC, MDSC subpopulation, or non-MDSC bound to the magnetic probe from an MDSC, MDSC subpopulation, or non-MDSC not bound to the magnetic probe; and (c) isolating the MDSC, MDSC subpopulation, or non-MDSC bound to the magnetic probe from the MDSC, MDSC subpopulation, or non-MDSC not bound to the magnetic probe. In some embodiments, applying the magnetic field results in the MDSC, MDSC subpopulation, or non-MDSC bound to a magnetic probe attaching to a magnetic bead. In some embodiments, the magnetic bead is a Dynabead®. In some embodiments, the bead is coated with an antibody or antigen-binding fragment thereof, lectin, enzyme, or streptavidin. In some embodiments, the magnetic probe is removed from the MDSC, MDSC subpopulation, or non-MDSC after the isolating.

In some embodiments, the MDSC, MDSC subpopulation, or non-MDSC is identified using an ELISA, multiplex immunoassay, western blot, or protein microarray. In some embodiments, the ELISA, multiplex immunoassay, western blot, or protein microarray comprises the use of an antibody or antigen-binding fragment thereof or an aptamer described herein to detect the MDSC, MDSC subpopulation, or non-MDSC. In some embodiments, the western blot is done after electrophoretic separation. In some embodiments, the ELISA is done without electrophoretic separation. In some embodiments, the ELISA, the multiplex immunoassay, the western blot, or the protein microarray provides a qualitative biomarker assessment, quantitative biomarker assessment, or combination thereof. In some embodiments, the ELISA, the multiplex immunoassay, the western blot, or the protein microarray are carried out on a cell lysate originating from MDSCs, an MDSC subpopulation, non-MDSCs, or a combination thereof.

In some embodiments, a level of a biomarker identifying the MDSC, MDSC subpopulation, or non-MDSC is quantified using real-time PCR (qRT-PCR). In some embodiments, the level of the biomarker is an expression level (e.g., an absolute or relative expression level).

In some embodiments, the MDSC, MDSC subpopulation, or non-MDSC is identified using sequencing. In some embodiments, a biomarker identifying the MDSC, a biomarker identifying the MDSC subpopulation, a biomarker identifying a non-MDSC, or a combination thereof are identified using sequencing. In some embodiments, DNA or RNA is sequenced. In some embodiments, the RNA is messenger RNA (mRNA). In some embodiments, mRNA is converted to complementary DNA (cDNA) prior to sequencing. In some embodiments the whole genome, the exome, or the transcriptome are evaluated or quantified by sequencing. In some embodiments, the DNA or RNA encoding the biomarker identifying the MDSC, MDSC subpopulation, or non-MDSC is sequenced. In some embodiments, sequencing includes Sanger sequencing, next generation sequencing (NGS), or a combination thereof. In some embodiments, next generation sequencing comprises massively-parallel signature sequencing, pyrosequencing (e.g., using a Roche 454 sequencing device), Illumina (Solexa) sequencing, sequencing by synthesis (Illumina), Ion torrent sequencing, sequencing by ligation (e.g., SOLiD sequencing), single molecule real-time (SMRT) sequencing (e.g., Pacific Bioscience), polony sequencing, DNA nanoball sequencing, heliscope single molecule sequencing (Helicos Biosciences), and/or nanopore sequencing (e.g., Oxford Nanopore).

In some embodiments, non-MDSCs are removed from a biological sample by removing cells which express a high level of Siglec-8, a high level of CD123, a high level of CD3, a high level of CD19, a high level of CD56, or a combination thereof.

In some embodiments, MDSCs or a subpopulation of MDSCs are isolated by selecting cells with a low level of Siglec-9, a low level of CD16, or a combination thereof. In some embodiments, MDSCs or a subpopulation of MDSCs are isolated by selecting cells with a low level of CD14, a high level of CD15, a low level of Siglec-9, a low level of CD16, or a combination thereof. In some embodiments, MDSCs or a subpopulation of MDSCs are isolated by selecting cells with a low level of CD14, a high level of CD15, a low level of Siglec-9, a low level of CD16, a low level of Siglec-5, a high level of Siglec-3, or a combination thereof. In some embodiments, MDSCs or a subpopulation of MDSCs are isolated by selecting cells with a low level of CD16, a low level of Siglec-9, a low level of Siglec-5, a high level of Siglec-3, or a combination thereof.

In some embodiments, a high level or a low level indicates a high level of expression of the biomarker on a surface of the cell or a low level of expression of the biomarker on a surface of the cell, respectively. As used herein, the superscripts or descriptors “+” and “high” are used interchangeably. As used herein, in certain embodiments, a high level of a biomarker is indicated with a “+,” for example CD15⁺. As used herein, in certain embodiments, a high level of a biomarker is indicated with a “high,” for example CD15^{high}. As used herein, the superscripts or descriptors “−” and “low” are used interchangeably. As used herein, in certain embodiments, a low level of a biomarker is indicated with a “−,” for example CD14⁻. As used herein, in certain embodiments, a low level of a biomarker is indicated with a “low,” for example CD14^{low}. In some embodiments, a low level of expression of the biomarker is no expression of the biomarker. In some embodiments, a low level of expression of the biomarker is a level of expression below a threshold level of expression. In some embodiments, a high level of expression of the biomarker is a level of expression above a threshold level of expression. In some embodiments, the threshold level of expression is a predetermined level of expression. In some embodiments, the threshold level of expression is a level of expression of non-cancerous cells in the individual from which the biological sample was taken. In some embodiments, the threshold level of expression is a level of expression in a healthy individual. In some embodiments, a low level of expression of the biomarker is a level of expression in a cell or cell population that is relatively lower or decreased compared to the level of biomarker expression in another cell or cell population from the same cellular or biological sample. In some embodiments, a low level of expression of the biomarker is a level of expression in a cell or cell population that is relatively lower or decreased compared to the level of biomarker expression in a cell or cell population from a different cellular or biological sample. In some embodiments, a high level of expression of the biomarker is a level of expression in a cell or cell population that is relatively higher or increased compared to the level of biomarker expression in another cell or cell population from the same cellular or biological sample. In some embodiments, a high level of expression of the biomarker is a level of expression in a cell or cell population that is relatively higher or increased compared to the level of biomarker expression in a cell or cell population from a different cellular or biological sample.

In some embodiments, methods of identifying an MDSC are used to diagnose a cancer in an individual. In some embodiments, diagnosing a cancer comprises identifying a subpopulation of MDSCs associated with the cancer. In some embodiments, the subpopulation of MDSCs associated with the cancer is an MDSC subpopulation expressing a low level of Siglec-9, a low level of CD16, or a combination thereof. In some embodiments, the subpopulation of MDSCs associated with the cancer are MDSCs expressing a low level of CD14, a high level of CD15, a low level of Siglec-9, a low level of CD16, or a combination thereof. In some embodiments, the subpopulation of MDSCs associated with the cancer are MDSCs expressing a low level of CD14, a high level of CD15, a low level of Siglec-9, a low level of CD16, a low level of Siglec-5, a high level of Siglec-3, or a combination thereof. In some embodiments, the subpopulation of MDSCs associated with the cancer are MDSCs expressing a low level of CD16, a low level of Siglec-9, a low level of Siglec-5, a high level of Siglec-3, or a combination thereof. In some embodiments, the cancer is a cancer of the adrenal gland, bile duct (e.g., cholangiocarcinoma), bladder, blood (e.g., a leukemia, a lymphoma, multiple myeloma, acute myeloid leukemia, acute lymphoid leukemia, chronic myeloid leukemia, or chronic lymphoid leukemia), bone, brain, breast, cervix, colorectal system (e.g., colorectal cancer or colon cancer), esophagus, gallbladder, gastric system, head and neck, kidney, liver, lung, ovary, pancreas, prostate, reticuloendothelial system, salivary gland, skin (e.g., melanoma), small intestine, soft tissue, thymus, or uterus. In some embodiments, the cancer is a neuroendocrine tumor. In some embodiments, the cancer is a gastrointestinal stromal tumor. In some embodiments, the cancer is a sarcoma. In some embodiments, the cancer is pancreatic cancer. In some embodiments, the pancreatic cancer is a pancreatic adenocarcinoma. In some embodiments, the pancreatic cancer is a pancreatic endocrine tumor (PET).

In some embodiments, diagnosing a cancer in an individual comprises identifying MDSCs or a subpopulation of MDSCs associated with the cancer in the biological sample from the individual.

In some embodiments, diagnosing a cancer in an individual comprises: (a) determining an amount of MDSCs or a subpopulation of MDSCs associated with the cancer in a biological sample from the individual; and (b) comparing the amount to a threshold amount. In some embodiments, the threshold amount of MDSCs or the threshold amount of the subpopulation of MDSCs is an amount of the same cells from a non-cancerous tissue in the individual. In some embodiments, the threshold amount of MDSCs or the subpopulation of MDSCs is an amount of the same cells in a biological sample from a healthy subject. In some embodiments, an amount of MDSCs or a subpopulation of MDSCs associated with the cancer above the threshold amount diagnoses the individual as having the cancer. In some embodiments, an amount of MDSCs or a subpopulation of MDSCs above the threshold amount indicates a treatment should be administered to the individual. In some embodiments, an amount of MDSCs or a subpopulation of MDSCs above the threshold amount indicates a treatment should not be administered to the individual. In some embodiments, an amount of MDSCs or a subpopulation of MDSCs above the threshold amount indicates a first treatment should be administered to the individual and a second treatment should not be administered to the individual. In some embodiments, an amount of MDSCs or a subpopulation of MDSCs associated with the cancer below the threshold amount diagnoses the individual as not having the cancer. In some embodiments, an amount of MDSCs or a subpopulation of MDSCs below the threshold amount indicates the individual should be administered a treatment. In some embodiments, an amount of MDSCs or a subpopulation of MDSCs below the threshold amount indicates the individual should not be administered a treatment. In some embodiments, an amount of MDSCs or a subpopulation of MDSCs below the threshold amount indicates the individual should be administered a first treatment, and should not be administered a second treatment.

In some embodiments, diagnosing a cancer in an individual comprises: (a) determining a proportion of MDSCs or a subpopulation of MDSCs in a biological sample from the individual, where the proportion is relative to an amount of cells in a second population; and (b) comparing the proportion to a threshold proportion. In some embodiments, the second population is all the cells in the biological sample, a subpopulation of MDSCs not associated with the cancer in the biological sample, or the non-MDSCs in the biological sample. In some embodiments, the threshold proportion is a proportion from a non-cancerous tissue in the individual. In some embodiments, the threshold proportion is a proportion from a healthy subject. In some embodiments, a proportion above the threshold proportion diagnoses the individual as having the cancer. In some embodiments, a proportion of MDSCs or a subpopulation of MDSCs above the threshold amount indicates a treatment should be administered to the individual. In some embodiments, a proportion of MDSCs or a subpopulation of MDSCs above the threshold amount indicates a treatment should not be administered to the individual. In some embodiments, a proportion of MDSCs or a subpopulation of MDSCs above the threshold amount indicates a first treatment should be administered to the individual and a second treatment should not be administered to the individual. In some embodiments, a proportion below the threshold proportion diagnoses the individual as not having the cancer. In some embodiments, a proportion of MDSCs or a subpopulation of MDSCs below the threshold amount indicates the individual should be administered a treatment. In some embodiments, a proportion of MDSCs or a subpopulation of MDSCs below the threshold amount indicates the individual should not be administered a treatment. In some embodiments, a proportion of MDSCs or a subpopulation of MDSCs below the threshold amount indicates the individual should be administered a first treatment, and should not be administered a second treatment.

In some embodiments, the diagnosing further comprises determining the severity of the cancer in the individual.

In some embodiments, methods of identifying MDSCs or a subpopulation of MDSCs are used to monitor a response of a cancer to a therapy in an individual.

In some embodiments, monitoring the response of a cancer in an individual to a therapy comprises: (a) determining a first amount of a subpopulation of MDSCs associated with the cancer in a first biological sample from the individual; (b) determining a second amount of a subpopulation of MDSCs associated with the cancer in a second biological sample from the individual; and (c) comparing the first amount to the second amount. In some embodiments, a decreased second amount relative to the first amount indicates a positive response of the cancer to the therapy. In some embodiments, an increased second amount compared to the first amount indicates a negative response of the cancer to the therapy.

In some embodiments, monitoring the response of a cancer in an individual to a therapy comprises: (a) determining a first proportion of a subpopulation of MDSCs relative to a second population of cells in a first biological sample from the individual; (b) determining a second proportion of a subpopulation of MDSCs relative to a second population of cells in a second biological sample from the individual; and (c) comparing the first proportion to the second proportion. In some embodiments, the second population is a total amount of cells in the first or the second biological sample, an amount of MDSCs in a subpopulation of MDSCs not associated with the cancer in the first or the second biological sample, or an amount of non-MDSCs in the first or the second biological sample. In some embodiments, a decreased second proportion compared to the first proportion indicates a positive response of the cancer to the therapy. In some embodiments, an increased second proportion compared to the first proportion indicates a negative response of the cancer to the therapy.

In some embodiments, a positive response indicates the cancer is decreasing in severity, the cancer is decreasing in size, the therapy is effective, no change in the cancer, no progression of cancer stage, or a combination thereof. In some embodiments, detection of a positive response further comprises maintaining an administration of the therapy to the individual. In some embodiments, detection of a positive further comprises a modification of administration of the therapy to the individual. In some embodiments, the administration of the therapy to the individual is reduced. In some embodiments, the administration of the therapy to the individual is increased. In some embodiments, the administration of the therapy to the individual is stopped.

In some embodiments, a negative response indicates the cancer is increasing in severity, the cancer is increasing in size, the therapy is not effective, or a combination thereof. In some embodiments, a negative response to a therapy is a relapse, a recurrence, an increase in severity, a progression of cancer stage, or no change in the cancer. In some embodiments, detection of a negative response further comprises a modification of administration of the therapy to the individual. In some embodiments, administration of the therapy to the individual is increased. In some embodiments, increasing the administration of the therapy comprises increasing an amount of the therapy administered to the individual, a frequency the therapy is administered to the individual, or a combination thereof. In some embodiments, detection of a negative response further comprises administering a second therapy to the individual. In some embodiments, when a second therapy is administered to the individual, administration of a first therapy is stopped. In some embodiments, when a second therapy is administered to the individual, administration of a first therapy continues.

In some embodiments, the first biological sample is taken from the individual prior to beginning the therapy. In some embodiments, the second biological sample is taken from the individual prior to beginning the therapy. In some embodiments, the second biological sample is taken from the individual after administration of the therapy. In some embodiments, a time between the taking the first biological sample from the individual and the second biological sample from the individual is 1 day, 2 days, 3 days, 4 days, 5 days, 6 days, 1 week, 2 weeks, 3 weeks, 4 weeks, 1 month, 2 months, 3 months, 4 months, 5 months, 6 months, or 1 year. In some embodiments, the method further comprises taking a third, a fourth, a fifth, a sixth, a seventh, an eighth, a ninth, or a tenth biological sample.

In some embodiments, the monitoring is done over the course of the therapy of the cancer in the individual. In some embodiments, the monitoring is done about 1 day, 2 days, 3 days, 4 days, 5 days, 6 days, 1 week, 2 weeks, 3 weeks, 4 weeks, 1 month, 2 months, 3 months, 4 months, 5 months, 6 months, or 1 year after administration of a first dose of the therapy to the individual.

In some embodiments, the therapy is a chemotherapy, an immunotherapy drug, a hormone therapy, a stem cell transplant, a radiation, a surgery, a small molecule drug, an antibody or antigen-binding fragment thereof, a checkpoint inhibitor, a kinase inhibitor, an oncolytic viral therapy, a gene-editing therapy, a cellular therapy (e.g., a chimeric antigen receptor (CAR)-T cell or transgenic T cell therapy) or a combination thereof In some embodiments, the chemotherapy is Abraxane®, Gemzar®, Onivyde®, or Folfinrinox. In some embodiments, the chemotherapy is irinotecan, paclitaxel, gemictibine, flurouracil (5-FU), leucovorin, oxaliplatin, or a combination thereof.

Also disclosed herein, in some embodiments, is a method of treating a cancer in a patient in need thereof, comprising administering an anti-cancer therapy to the patient, wherein a biological sample from the patient has been identified as comprising a population of myeloid-derived suppressor cells (MDSCs) as disclosed herein.

In some embodiments, the cancer is a cancer of the adrenal gland, bile duct (e.g., cholangiocarcinoma), bladder, blood (e.g., a leukemia, a lymphoma, multiple myeloma, acute myeloid leukemia, acute lymphoid leukemia, chronic myeloid leukemia, or chronic lymphoid leukemia), bone, brain, breast, cervix, colorectal system (e.g., colorectal cancer or colon cancer), esophagus, gallbladder, gastric system, head and neck, kidney, liver, lung, ovary, pancreas, prostate, reticuloendothelial system, salivary gland, skin (e.g., melanoma), small intestine, soft tissue, thymus, or uterus. In some embodiments, the cancer is a neuroendocrine tumor. In some embodiments, the cancer is a gastrointestinal stromal tumor. In some embodiments, the cancer is a sarcoma. In some embodiments, the cancer is pancreatic cancer. In some embodiments, the pancreatic cancer is a pancreatic adenocarcinoma. In some embodiments, the pancreatic cancer is a pancreatic endocrine tumor (PET).

In some embodiments, the anti-cancer therapy is a chemotherapy, an immunotherapy drug, a hormone therapy, a stem cell transplant, a radiation, a surgery, a small molecule drug, an antibody or antigen-binding fragment thereof, a checkpoint inhibitor, a kinase inhibitor, an oncolytic viral therapy, a gene-editing therapy, a cellular therapy (e.g., a chimeric antigen receptor (CAR)-T cell or transgenic T cell therapy) or a combination thereof. In some embodiments, the chemotherapy is Abraxane®, Gemzar®, Onivyde®, or Folfinrinox. In some embodiments, the chemotherapy is irinotecan, paclitaxel, gemictibine, flurouracil (5-FU), leucovorin, oxaliplatin, or a combination thereof.

### Kits for Identification of Myeloid-Derived Suppressor Cells (MDSCs)

For use in the methods described herein, kits and articles of manufacture are also provided. In some embodiments, the kit comprises a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

In some embodiments, a kit comprises one or more additional containers, each with one or more of various materials (such as reagents, optionally in concentrated form, and/or devices) desirable from a commercial and user standpoint for use in a method described herein. Non-limiting examples of such materials include, but not limited to, buffers, diluents, detection agent, detectable probes, filters, needles, syringes; carrier, package, container, vial and/or tube labels listing contents and/or instructions for use, and package inserts with instructions for use. In some embodiments, a set of instructions is included. In some embodiments, a label is on or associated with the container. For example, a label is on a container when letters, numbers or other characters forming the label are attached, molded or etched into the container itself. In another example, a label is associated with a container when it is present within a receptacle or carrier that also holds the container, e.g., as a package insert. In some embodiments, a label is used to indicate that the contents are to be used for a specific diagnostic application. In some embodiments, the label indicates directions for use of the contents, such as in the methods described herein.

In some embodiments, the kit comprises at least one detectable probe capable of detecting a neutrophil biomarker, a monocyte biomarker, CD16, Siglec-9, or a combination thereof. In some embodiments, the neutrophil biomarker is CD15. In some embodiments, the monocyte biomarker is CD14. In some embodiments, the kit further comprises at least one detectable probe capable of detecting Siglec-5, Siglec-3, or a combination thereof. In some embodiments, the kit further comprises a detectable probe capable of detecting a non-MDSC biomarker. In some embodiments, the non-MDSC is an eosinophil, basophil, or lymphocyte. In some embodiments, an eosinophil biomarker is Siglec-8. In some embodiments, a basophil biomarker is CD123. In some embodiments, a lymphocyte biomarker is CD3, CD19, CD56, or a combination thereof

In some embodiments, the detectable probe comprises an antibody, antigen-binding fragment thereof, or an aptamer. In some embodiments, the antibody, antigen-binding fragment thereof, or aptamer binds to CD14, CD15, CD16, Siglec-9, Siglec-3, Siglec-5, Siglec-8, CD123, CD3, CD19, CD56, or a combination thereof. In some embodiments, the detectable probe is conjugated to a fluorophore. In some embodiments, the detectable probe is fluorescently detectable. In some embodiments, the detectable probe comprises a magnetic particle. Certain Terminology

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein are. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). Further, these terms refer to human or animal subjects.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### EXAMPLES

As used herein, when referring to the presence of a biomarker, the superscripts or descriptors “low” and “−” are used interchangeably, and indicate that a particular biomarker is present in amounts relatively lower in some cells as compared to other cells. Although the use of “low” or “−” does not necessarily man absent, in some situations, the use of “low” or “−” will include cells where the biomarker is absent. Likewise, as used herein, when referring to the presence of a biomarker, the superscripts or descriptors “high” and “+” are used interchangeably, and indicate that a particular biomarker is present in amounts relatively higher in some cells as compared to other cells.

### Example 1

### Myeloid Derived Suppressor Cells (MDSCs) are Increased in Pancreatic Cancer

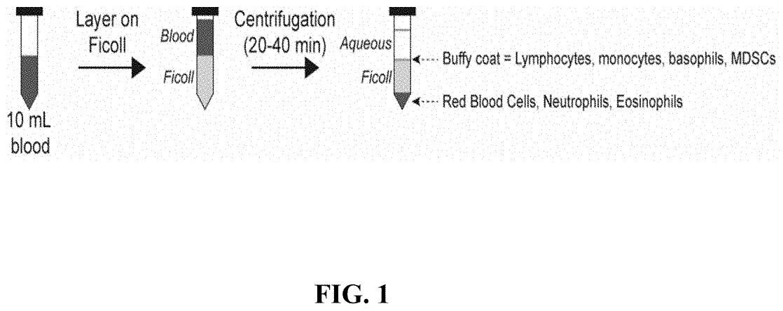

Myeloid derived suppressor cell (MDSC) populations from healthy individuals were analyzed from preparations of peripheral blood that include: (1) whole blood samples, (2) granulocyte samples, and (3) buffy coat samples. The granulocyte and buffy coat samples were prepared as generally outlined in FIG. 1. As shown in FIG. 1, MDSCs are typically co-purified with other low-density mononuclear cells in the buffy coat layer while higher density granulocytes (e.g., neutrophils and eosinophils) typically sediment in a layer with the red blood cells.

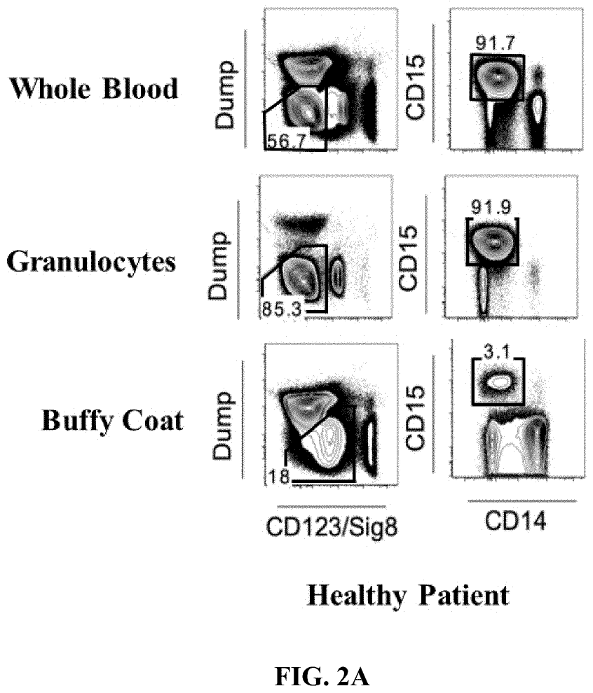

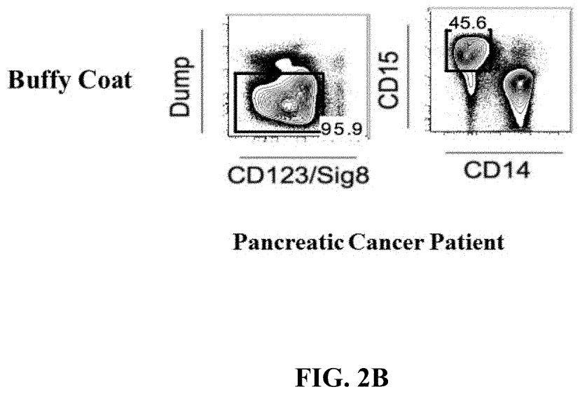

Cells from the whole blood, granulocyte, and buffy coat samples were stained with antibodies against CD3, CD19, and CD56 (to detect lymphocytes), CD123 (to detect basophils), Siglec-8 (to detect eosinophils), CD14 (to detect monocytes), and CD15 (to detect neutrophils) and subjected to flow cytometry analysis to detect MDSC populations. FIG. 2A provides representative results from a healthy individual. A three step gating strategy was utilized. First, a forward scatter (FSC) and side scatter (SSC) gate was placed broadly to capture “live” cells (not shown). Second, cells with low levels of CD3, CD19, and CD56 (“Dump,” Y-axis FIG. 2A, left panels) and low levels of CD123 and Siglec-8 (“CD123/Sig8,” X-axis FIG. 2A, left panels) were gated to remove lymphocytes, basophils, and eosinophils. As a result, 56.7% of cells in the whole blood sample, 85.3% of cells in the granulocyte sample, and 18% of cells in the buffy coat sample carried forward to the third stage of analysis. To quantify cells indicative of MDSCs, cells were gated again to select for cells with low levels of CD14 “CD14^{low}” (X-axis FIG. 2A, right panels) and high levels of CD15 “CD15^{high}” (Y-axis FIG. 2A, right panels). As shown in FIG. 2A, after gating out lymphocytes, basophils, and eosinophils, 91.7% of remaining cells in the whole blood sample (˜52.1% overall), 91.9% of remaining cells in the granulocyte sample (˜78.4% overall), and 3.1% of remaining cells in the buffy coat sample (˜0.5% overall) were captured by the CD15^{high}/CD14^{low} gate. Thus, while the gating strategy described above indicates the detection of MDSCs in a buffy coat sample, such methods fail to provide meaningful results with whole blood samples, where detection of MDSCs is obscured by the relatively large population of neutrophils present in the sample.

To compare the relative amounts of MDSCs in healthy patients to MDSCs in cancer patients, buffy coat samples from pancreatic cancer patients were prepared generally as outlined in FIG. 1 and subjected to flow cytometry analysis as described above. As seen in FIG. 2B, cells from a representative pancreatic cancer patient with low levels of CD3, CD19, and CD56 (“Dump” Y-axis FIG. 2B) as well as low levels of CD123, and Siglec-8 (“CD123/Sig8” X-axis FIG. 2B, left panel) were gated, resulting in approximately 95.9% of cells carried forward for further analysis. To quantify MDSCs, cells were gated again (“CD15^{high}/CD14^{low}”) to select for cells with low levels of CD14 (“CD14” X-axis, FIG. 2B, right panel) and high levels of CD15 (“CD15” Y-axis, FIG. 2B, right panel). As shown in FIG. 2B, after gating out lymphocytes, basophils, and eosinophils, 45.6% of remaining cells in the buffy coat sample were present in the CD15^{high}/CD14^{low} gate and indicative of the presence of MDSCs. Overall, the gating strategy described above demonstrated that approximately 40% of all cells detected in a buffy coat sample of a representative pancreatic cancer patient were indicative of the presence of MDSCs (compare to the results shown in FIG. 2A for a buffy coat sample of a representative healthy patient, where only ˜0.5% of all cells detected were indicative of MDSCs).

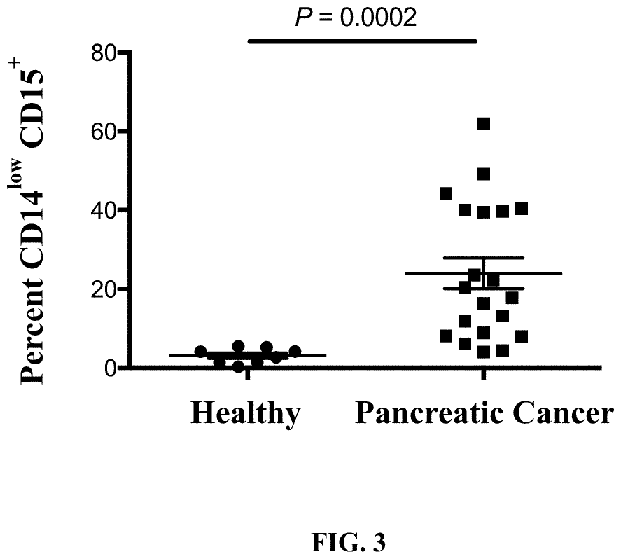

To further characterize MDSCs in pancreatic cancer patients, buffy coat samples from healthy individuals and pancreatic cancer patients were analyzed by flow cytometry to quantify MDSCs using the gating strategy as described above. As seen in FIG. 3, MDSC cell populations from buffy coat samples of pancreatic cancer patients were observed to be dramatically and significantly increased (P=0.0002) as compared to MDSC cell populations detected in buffy coat samples from healthy individuals.

### Example 2

### Detection and Characterization of CD16⁻ MDSC Subpopulations

Myeloid derived suppressor cell (MDSC) populations from a representative healthy individual and a representative pancreatic cancer patient were analyzed from preparations of peripheral blood that include: (1) whole blood samples, (2) granulocyte samples, and (3) buffy coat samples. The granulocyte and buffy coat samples were prepared generally as described in Example 1.

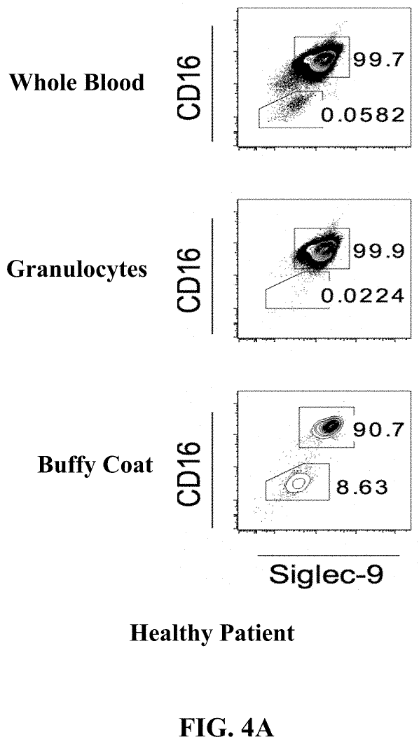

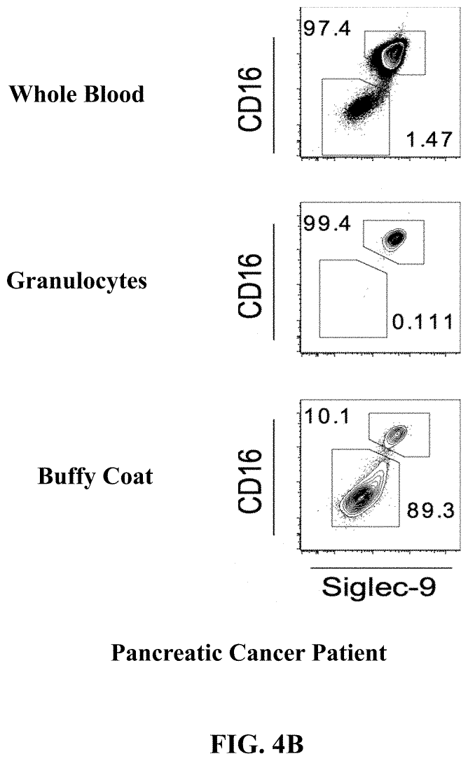

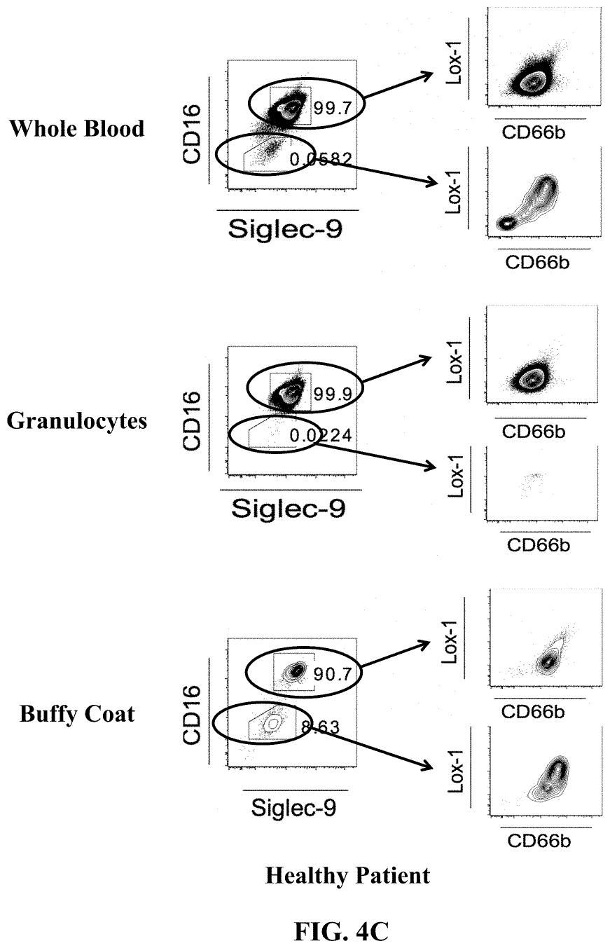

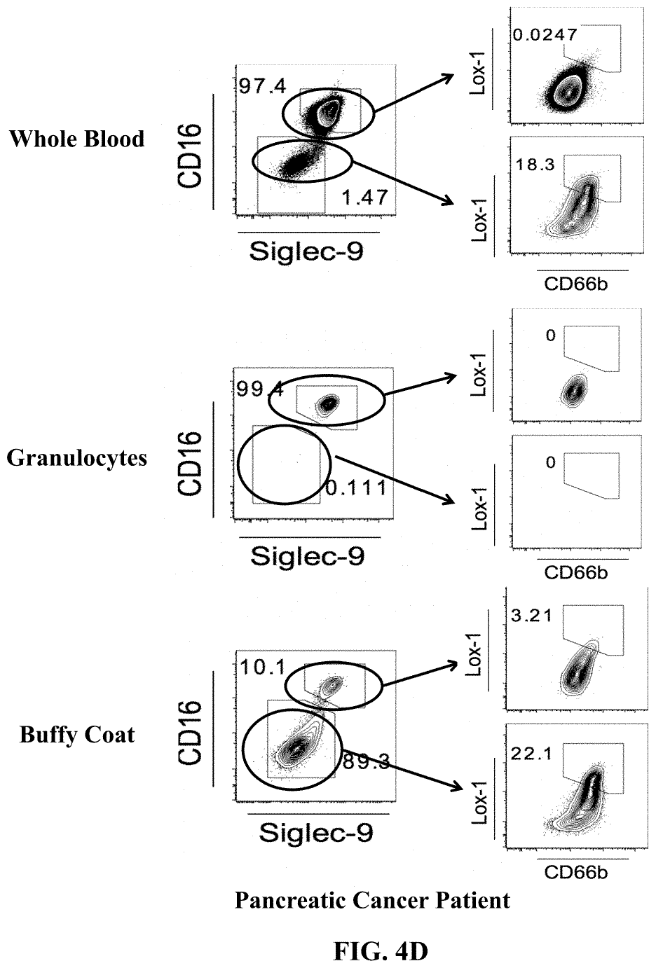

Cells from whole blood, granulocyte, and buffy coat samples were stained with antibodies against CD16 and Siglec-9 in addition to the antibodies described in Example 1 (CD3, CD19, CD56, CD123, Siglec-8, CD14, and CD15) and subjected to flow cytometry analysis and gating as described in Example 1. The CD15^{high}/CD14^{low} MDSCs cells were then further analyzed to characterize the level of CD16 and Siglec-9 in these cells. As shown in FIGS. 4A-4B, for both the healthy individual and the pancreatic cancer patient, CD15^{high}/CD14^{low} MDSCs can be further subdivided into two distinct subpopulations: (1) a first subpopulation with low levels of CD16 (“CD16⁻”) and low levels of Siglec-9 (“Sig9⁻”); and (2) a second subpopulation with relatively higher levels of CD16 (“CD16⁺”) and relatively higher levels of Siglec-9 (“Sig9⁺”). As seen in FIG. 4A, for the healthy individual, ˜0.06% of cells in the whole blood sample, ˜0.02% of cells in the granulocyte sample, and ˜8.63% of cells in the buffy coat sample were captured by the gate and indicative of a CD16⁻/Sig9⁻ subpopulation of MDSCs. On the other hand, as seen in FIG. 4B, ˜1.47% of cells in the whole blood sample, ˜0.11% of cells in the granulocyte sample, and ˜89.3% of cells in the pancreatic cancer patient buffy coat sample were captured by the gate and indicative of a CD16⁻/Sig-9⁻ subpopulation of MDSCs. Thus, overall, there was a more than 10-fold increase in the CD16⁻/Sig9⁻ subpopulation of MDSCs in the pancreatic cancer patient, suggesting that this subpopulation of MDSCs may play a prominent role in the MDSC-mediated suppression of T-cell activation in pancreatic cancer.

Analysis of LOX-1 in CD16^{low}/CD16^{high} MDSC Subpopulations

Recently, Condamine, et al., reported that lectin-type oxidized LDL receptor-1 (LOX-1) was one of the most overexpressed cell-surface proteins in CD11b⁺CD14⁻CD15⁺/CD66b⁺ polymorphonuclear (granulocyte-type) myeloid-derived suppressor cells (PMN-MDSCs) and that LOX-1 could potentially distinguish PMN-MDSCs from neutrophils, which generally lack LOX-1 expression. See, Condamine, et al., Sci. Immunol. Aug. 5, 2016; 1(2):aaf8943. To examine the expression of LOX-1 in CD16⁺/CD16⁻ MDSC subpopulations in both healthy individuals and in pancreatic cancer patients, cells from whole blood, granulocyte, and buffy coat samples from were stained with antibodies against LOX-1, CD66b, CD16, and Siglec-9 in addition to the antibodies described in Example 1 (CD3, CD19, CD56, CD123, Siglec-8, CD14, and CD15). These cells were then subjected to flow cytometry analysis and gating as described in Example 1. The CD15^{high}/CD14^{low} MDSCs cells were then gated to distinguish the CD16^{low} and CD16^{high} MDSC subpopulations as previously described. The CD16^{low}/Siglec-9^{low} and CD16^{high}/Siglec-9^{high} MDSC subpopulations were then further analyzed to characterize the level of CD66b and LOX-1 in these cells.

As shown in FIG. 4C and FIG. 4D, higher levels of LOX-1 expression inversely correlated with the levels of CD16 expression when examined in either a representative healthy individual (FIG. 4C) or pancreatic cancer patient (FIG. 4D). For example, in the pancreatic cancer samples, only ˜0.02% of CD16^{high} cells from the whole blood sample and ˜3.21% of CD16^{high} cells from the buffy coat sample exhibited increased levels of LOX-1 expression (FIG. 4D). In contrast, ˜18.3% of CD16^{low} cells from the whole blood sample and ˜22.1% of CD16^{low} cells from the buffy coat sample exhibited high levels of LOX-1 expression (FIG. 4D). Accordingly, the LOX-1⁺ MDSC subpopulation described by Condamine, et al. is almost exclusively found within the CD16^{low} MDSC subpopulation described herein. However, as shown in the whole blood and buffy coat samples of FIGS. 4C-4D, LOX-1 staining did not result in the separation of MDSCs into discrete LOX-1^{low} and LOX-1^{high} subpopulations, but instead was observed as a continuum of LOX-1 expressing cells ranging from relatively lower to relatively higher levels of LOX-1 (contrast to CD16 staining observed in FIGS. 4C-4D, which demonstrates clear separation of MDSCs into distinct CD16^{low} and CD16^{high} subpopulations). Thus, LOX-1 is not likely to be a very effective marker for detecting MDSCs from whole blood because the LOX-1 signal is not very intense, and as seen in FIGS. 4C-4D, is not able to effectively distinguish neutrophils from MDSCs in whole blood samples.

### Example 3

### Siglec-3, Siglec-5, and Siglec-9 Exhibit Altered Expression in CD16^{low} MDSCs

Based upon the increased numbers of CD16^{low}/Sig9^{low} MDSCs observed by flow cytometry in pancreatic cancer patients, peripheral blood was collected from another cohort of healthy and pancreatic cancer patients to more precisely and quantitatively determine the CD16^{low}/Sig9^{low} MDSC subpopulations. Whole blood samples were stained with antibodies against CD16, Siglec-3, Siglec-5, and Siglec-9 in addition to the antibodies described in Example 1 (CD3, CD19, CD56, CD123, Siglec-8, CD14, and CD15) and subjected to flow cytometry analysis and gating as described in Example 1 to select for CD15^{high}/CD14^{low} MDSCs. The CD15^{high}/CD14^{low} MDSCs were then further gated for MDSC subpopulations with low levels of CD16 (“CD16^{low}”).

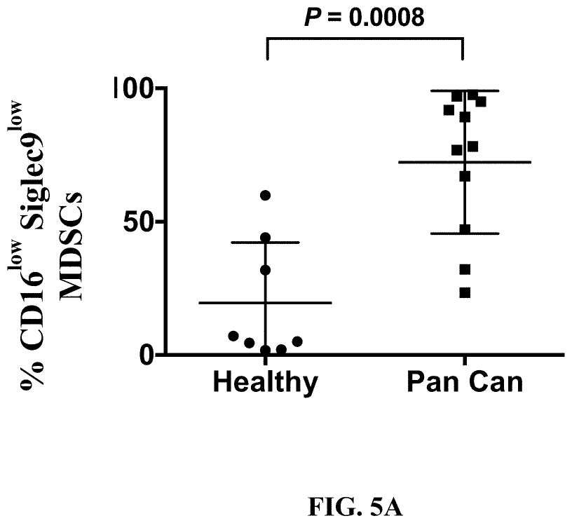

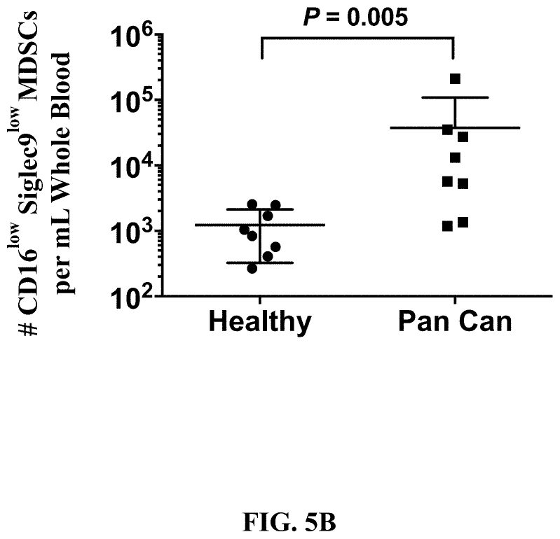

As shown in FIG. 5A, CD16^{low}/Sig-9^{low} MDSCs were observed at a significantly higher percentage of CD15^{high}/CD14^{low} MDSC populations (P=0.0008) in individuals with pancreatic cancer (“Pan Can”) compared to healthy individuals (“Healthy”). As seen in FIG. 5B, significantly increased numbers of CD16^{low}/Siglec-9^{low} MDSCs (P=0.005) were detected per mL of whole blood in individuals with pancreatic cancer (“Pan Can”) compared to healthy individuals (“Healthy”). Thus, not only do CD16^{low}/Siglec-9^{low} MDSCs represent a higher proportion of the overall MDSC population in pancreatic cancer patients (FIG. 5A), but they are also observed in far greater numbers in pancreatic cancer patients compared to healthy individuals.

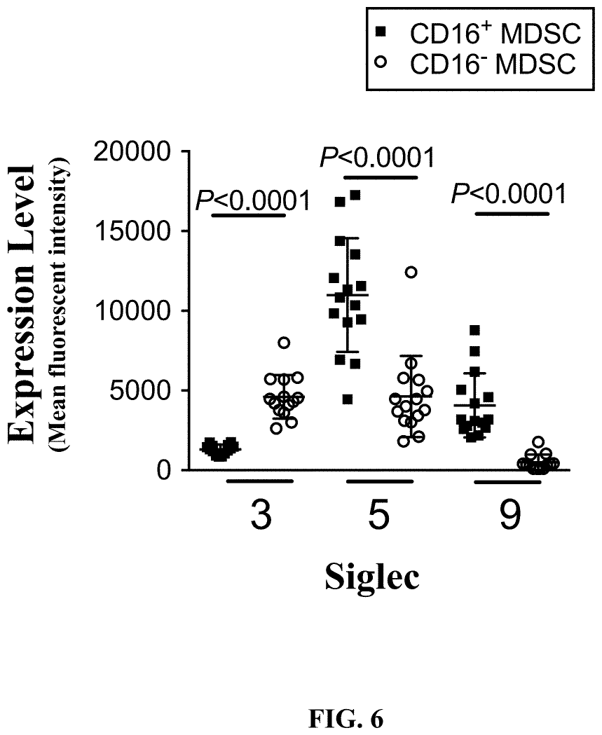

To further characterize the expression of Siglec family members in CD16^{low} MDSCs derived from pancreatic cancer patients, buffy coat samples from pancreatic cancer patients were stained as previously described to select for CD15^{high}/CD14^{low} MDSCs. The CD15^{high}/CD14^{low} MDSCs were then further gated for MDSC subpopulations with low levels of CD16 (“CD16⁻”) and high levels of CD16 (“CD16⁺”). Siglec-3, Siglec-5, and Siglec-9 expression levels were then quantitated (mean fluorescent intensity) in the CD16⁺and CD16⁻ MDSC subpopulations. As seen in FIG. 6, expression levels of Siglec-3 were increased in CD16⁻ vs. CD16⁺ MDSCs (P<0.0001), while both Siglec-5 and Siglec-9 were decreased in CD16⁻ vs. CD16⁺ MDSCs (P<0.0001). Interestingly, the expression levels of Siglecs-3, -5, and -9 observed in the CD16⁺ MDSC subpopulation were exactly the same as Siglec expression levels observed in neutrophils (data not shown), indicating a challenge in differentiating neutrophils from MDSCs in whole blood samples.

Overall, while the biomarkers and stains described in Example 1 are capable of detecting MDSC populations from a buffy coat preparation, they are not particularly useful for identifying MDSC populations from whole blood samples. However, as shown herein, the additional selection of cells for low levels of CD16 and low levels of Siglec-9 allows for the detection of a subpopulation of MDSCs from whole blood samples that is distinct from neutrophils and significantly upregulated in cancer patients. Accordingly, techniques or devices using the biomarkers described above have significant advantages over existing techniques or devices by increasing the accuracy and precision of measuring or isolating MDSCs.

### Example 4

### T-Cell Proliferation in Mixed Lymphocyte Reactions with CD16⁻ Versus CD16⁺ MDSCs

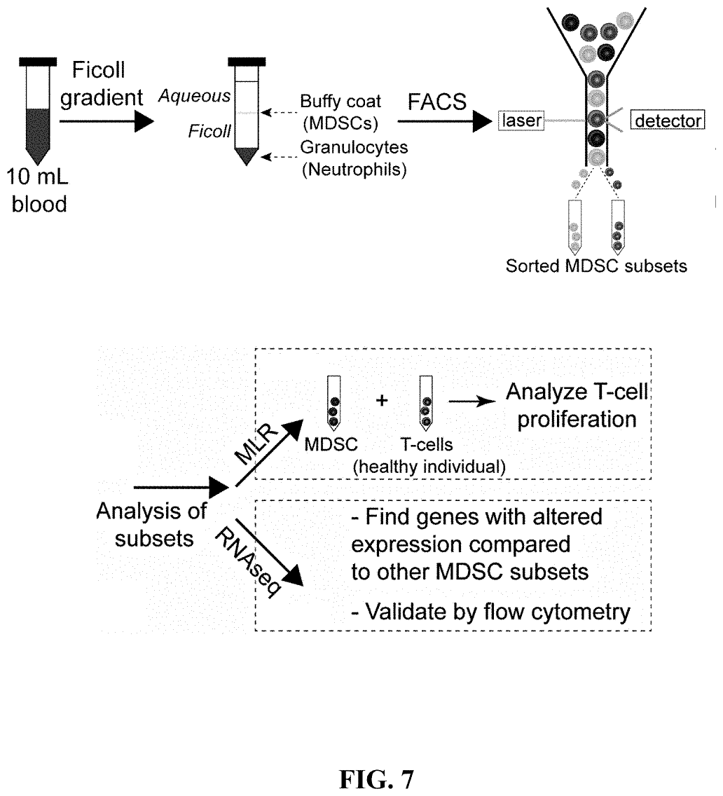

Cells isolated from buffy coat peripheral blood samples from either healthy or pancreatic cancer patients were stained with antibodies against CD3, CD19, CD56, CD123, Siglec-8, CD14, CD15, and CD16 as previously described and FACS sorted for subsequent analysis as generally illustrated in FIG. 7. Pancreatic cancer polymorphonuclear cells (granulocytes) were also harvested generally as described in Example 1. CD4⁺and CD8⁺ T-cells from a healthy individual were magnetically enriched, fluorescently labeled with Cell Trace Violet, and used in a one-way mixed lymphocyte reaction (“MLR”) to measure CD8⁺ or CD4⁺ T-cell proliferation in response to CD15⁺/CD14⁻/CD16⁺(“CD16⁺”) or CD15⁺/CD14⁻/CD16⁻(“CD16⁻”) MDSCs.

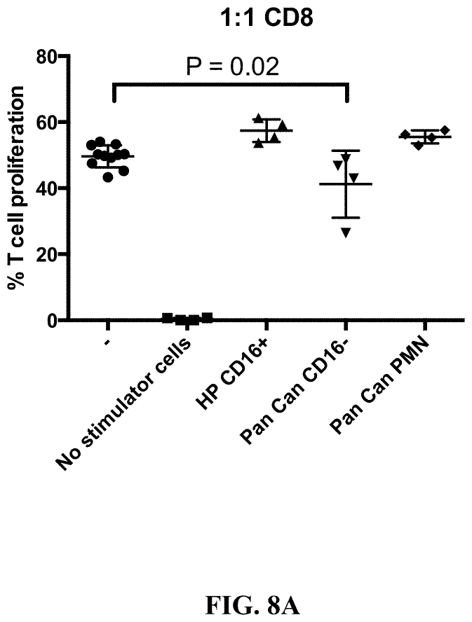

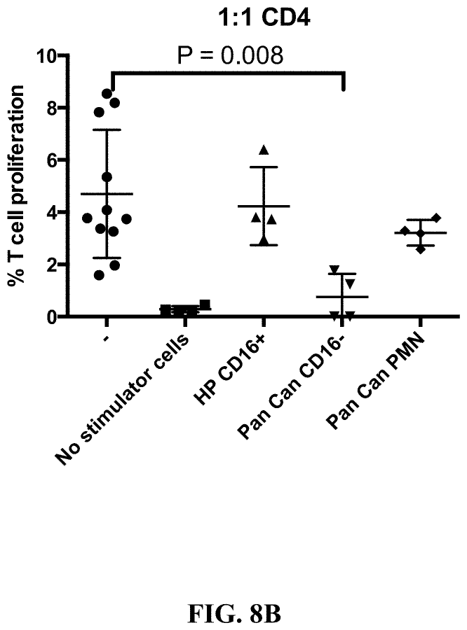

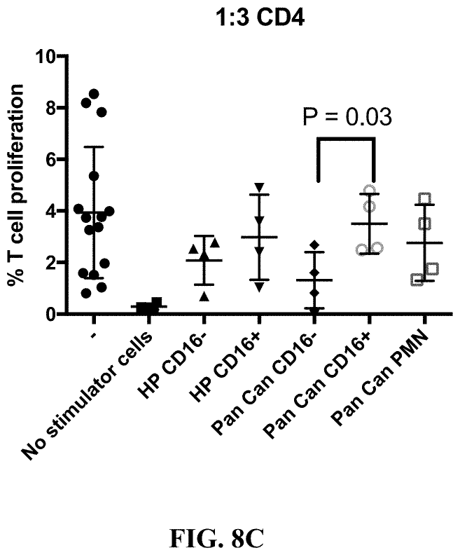

CD8⁺ T-cells were mixed in a 1:1 ratio with healthy CD16⁺ MDSCs, pancreatic cancer CD16⁻ MDSCs, or pancreatic cancer granulocytes in culture for 5 days and T-cell proliferation rates were measured by fluorescence dilution of the labeled CD8⁺ T-cells. As seen in FIG. 8A, the proliferation rate of CD8⁺ T-cells incubated with pancreatic cancer CD16⁻MDSCs (“Pan Can CD16⁻”) was significantly reduced (P=0.02) compared to CD8⁺ T-cells and stimulator cells with no MDSCs (“-”). The proliferation rate of CD8⁺ T-cells was unaffected by CD16⁺ MDSCs from a healthy patient (“HP CD16⁺”) or granulocytes from a pancreatic cancer patient (“Pan Can PMN”).

CD4⁺ T-cells were mixed in a 1:1 ratio with healthy CD16⁺ MDSCs, pancreatic cancer CD16⁻ MDSCs, or pancreatic cancer granulocytes and T-cell proliferation rates were measured by fluorescence dilution of the labeled CD4⁺ T-cells. As seen in FIG. 8B, the proliferation rate of CD4⁺ T-cells incubated with pancreatic cancer CD16⁻ MDSCs (“Pan Can CD16⁻”) was significantly reduced (P=0.008) compared to CD4⁺ T-cells with no MDSCs (“−”). The proliferation rate of CD4⁺ T-cells was unaffected by CD16⁺ MDSCs from a healthy patient (“HP CD16⁺”) or granulocytes from a pancreatic cancer patient (“Pan Can PMN”).

In a separate experiment, CD4⁺ T-cells were mixed in a 1:3 ratio with CD16⁺ MDSCs from a healthy patient, CD16⁻ MDSCs from a healthy patient, CD16⁺ MDSCs from a pancreatic cancer patient, CD16⁻ MDSCs from a pancreatic cancer patient, or granulocytes from a pancreatic cancer patient, and T-cell proliferation rates were measured by fluorescence dilution of the labeled CD4⁺ T-cells. As seen in FIG. 8C, the proliferation rate of CD4⁺ T-cells incubated with pancreatic cancer CD16⁻ MDSCs (“Pan Can CD16⁻”) was significantly reduced (P=0.03) compared to CD4⁺ T-cells incubated with CD16⁺ MDSCs from a pancreatic cancer patient (“Pan Can CD16⁺”).

Overall, CD16⁻ MDSCs from pancreatic cancer patients were found to be suppressive of both CD8⁺ and CD4⁺ T-cells, indicating that CD16⁻ MDSCs likely contribute to the immunosuppressive tumor microenvironment observed in many forms of cancer.

### Example 5

### Next Generation Sequencing of MDSC Populations

Distinct MDSC subpopulations (such as CD15⁺/CD14⁻/CD16⁺(“CD16⁺”) or CD15⁺/CD14⁻/CD16⁻(“CD16⁻”) MDSCs) are isolated from buffy coat peripheral blood samples (from, e.g., cancer patients) and FACS sorted for subsequent analysis as generally illustrated in FIG. 7. Nucleic acids present in isolated MDSC cell subpopulations are then subjected to next generation sequencing (e.g., whole transcriptome RNA sequencing to identify altered mRNA transcripts or whole genome/exome sequencing to identify, e.g., SNPs, CNVs, and/or DNA rearrangement events) to identify biomarkers useful for the detection and treatment of MDSC-influenced cancers. Potential novel biomarkers discovered through next-generation sequencing are then validated as generally described herein (e.g., by flow cytometry).

### Example 6

### Monitoring Treatment of a Pancreatic Cancer Patient

A 60-year old man suffering from a pancreatic adenocarcinoma begins a chemotherapeutic treatment for his cancer comprising gemcitabine. Prior to beginning chemotherapy, a first blood sample is taken and the quantity of CD15⁺/CD14⁻/Siglec-9⁻/CD16⁻MDSCs is determined as previously described herein. After 4 weeks of chemotherapy, a second blood sample is taken and the quantity of CD15⁺/CD14⁻/Siglec-9⁻/CD16⁻ MDSCs is determined. An increase in the amount or relative proportion of CD15⁺/CD14⁻/Siglec-9⁻/CD16⁻ MDSCs from the first blood sample to the second blood sample indicates that the gemcitabine is not effective. In response, the chemotherapeutic agent is changed from gemcitabine to a cocktail of drugs comprising 5-FU/leucovorin, irinotecan, and oxliplatin.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-152. (canceled)

153. A method of identifying a set of myeloid-derived suppressor cells (MDSCs) in a population of cells of a biological sample, comprising: detecting amounts of (i) a neutrophil biomarker, (ii) a monocyte biomarker, (iii) CD16, and (iv) Siglec-9 of each cell of the population of cells, wherein the set of MDSCs comprise (i) high levels of the neutrophil biomarker; (ii) low levels of the monocyte biomarker; (iii) low levels of the CD16; and (iv) low levels of the Siglec-9.

154. The method of claim 153, wherein the neutrophil biomarker is CD15 and the monocyte biomarker is CD14.

155. The method of claim 153, further comprising detecting amounts of: Siglec-5, CD33 (Siglec-3), an eosinophil biomarker, a basophil biomarker, a lymphocyte biomarker, or a combination thereof, wherein the set of MDSCs comprise low levels of Siglec-5, high levels of CD33 (Siglec-3), low levels of the eosinophil biomarker, low levels of the basophil biomarker, low levels of the lymphocyte biomarker, or a combination thereof.

156. The method of claim 155, wherein the eosinophil biomarker is Siglec-8, the basophil biomarker is CD123, and the lymphocyte biomarker comprises CD3, CD19, CD56, or a combination thereof.

157. The method of claim 153, wherein the biological sample is a tissue sample, a blood sample, a whole blood sample, a granulocyte sample or a buffy coat sample.

158. The method of claim 153, wherein said detecting amounts of (i) the neutrophil biomarker, (ii) the monocyte biomarker, (iii) CD16, and (iv) Siglec-9 of each cell of the population of cells comprises using flow cytometry.

159. The method of claim 153, wherein the method further comprises detecting a cancer.

160. The method of claim 159, wherein the cancer is selected from the list consisting of: a pancreatic cancer, a lung cancer, a colon cancer, a breast cancer, a gastric cancer, an esophageal cancer, an ovarian cancer, a uterine cancer, a prostate cancer, a bladder cancer, a liver cancer, a cholangiocarcinoma, a neuroendocrine tumor, a gastrointestinal stromal tumor, a sarcoma, a brain cancer, a skin cancer, a melanoma, a liquid tumor, a multiple myeloma, an acute myeloid leukemia, an acute lymphoid leukemia, a chronic myeloid leukemia, and a chronic lymphoid leukemia.

161. The method of claim 153, wherein the biological sample is from a patient diagnosed with cancer or suspected of having cancer.

162. The method of claim 161, wherein the patient previously received an anti-cancer therapy.

163. A kit for detecting myeloid-derived suppressor cells (MDSCs) comprising a neutrophil biomarker labelling agent, a monocyte biomarker labelling agent, a CD16 labelling agent, and a Siglec-9 labelling agent.

164. The kit of claim 163, further comprising one or more agents selected from the group consisting of: a Siglec-5 labelling agent, a CD33 (Siglec-3) labelling agent, an eosinophil biomarker labelling agent, a basophil biomarker labelling agent, and a lymphocyte biomarker labelling agent.

165. The kit of claim 164, wherein:
a. the monocyte biomarker labelling agent comprises an antibody or antigen-binding fragment thereof that binds to CD14;
b. the neutrophil labelling agent comprises an antibody or antigen binding fragment thereof that binds to CD15;
c. the CD16 labelling agent comprises an antibody or antigen-binding fragment thereof that binds to CD16;
d. the Siglec-9 labelling agent comprises an antibody or antigen-binding fragment thereof that binds to Siglec-9;
e. the Siglec-5 labelling agent comprises an antibody or antigen-binding fragment thereof that binds Siglec-5;
f. the CD33 (Siglec-3) labelling agent comprises an antibody or antigen-binding fragment thereof that binds to CD33 (Siglec-3);
g. the eosinophil labelling agent biomarker comprises an antibody or antigen-binding fragment thereof that binds to Siglec-8;
h. the basophil biomarker labelling agent comprises an antibody or antigen-binding fragment thereof that binds to CD123;
i. the lymphocyte biomarker labelling agent comprises an antibody or antigen-binding fragment thereof that binds to CD3;
j. the lymphocyte biomarker labelling agent comprises an antibody or antigen-binding fragment thereof that binds to CD19;
k. the lymphocyte biomarker labelling agent comprises an antibody or antigen-binding fragment thereof that binds to CD56;
or a combination thereof.

166. A method of treating a cancer in a patient in need thereof, comprising:
a) ascertaining that a biological sample from the patient comprises a population of myeloid-derived suppressor cells (MDSCs) comprising:
(i) high levels of a neutrophil biomarker;
(ii) low levels of a monocyte biomarker;
(iii) low levels of CD16; and
(iv) low levels of Siglec-9, and
b) administering an anti-cancer therapy to the patient.

167. The method of claim 166, wherein the anti-cancer therapy is a modified anti-cancer therapy.

168. A method of monitoring anti-cancer therapy in a subject in need thereof comprising:
a) quantifying cells comprising
(i) high levels of a neutrophil biomarker;
(ii) low levels of a monocyte biomarker;
(iii) low levels of CD16; and
(iv) low levels of Siglec-9 in a first sample from the subject, and
b) quantifying cells comprising
(i) high levels of a neutrophil biomarker;
(ii) low levels of a monocyte biomarker;
(iii) low levels of CD16; and
(iv) low levels of Siglec-9 in a second sample from the subject,
wherein the first sample was collected before the second sample.

169. The method of claim 168, further comprising, recommending a change in the anti-cancer therapy if the quantity of (b) is larger than the quantity of (a).

170. The method of claim 168, wherein the subject received an anti-cancer therapy before the first sample was obtained.
